(19) United States
(12) Patent Application Publication
(10) Pub. No.: US 2021/0000980 A1
(43) Pub. Date: Jan. 7, 2021

(54) METHOD AND COMPOSITION FOR THE DETECTION OF ARTHROPODS

(71) Applicant: ARKE' S.r.l., Pomezia (RM) (IT)

(72) Inventors: Veronica OJETTI, Roma (RM) (IT); Valentina CICCONE, Roma (RM) (IT)

(21) Appl. No.: 16/968,666

(22) PCT Filed: Feb. 15, 2019
(86) PCT No.: PCT/IB2019/051240

§ 371 Date: Aug. 10, 2020

(30) Foreign Application Priority Data
Feb. 16, 2018 (IT) 102018000002747

(51) Int. Cl. A61K 49/00; A61K 36/61

(52) U.S. Cl. CPC A61K 49/0021; A61K 36/61; A61K 49/0089; A61K 49/0054

(57) ABSTRACT

The present invention relates to a method and a composition for detecting the presence of arthropods, and/or their eggs, in the hair of the body and/or head of a subject.

The present invention relates to a method and a composition for detecting the presence of arthropods, and/or their eggs, in the hair of the body and/or head of a subject.

An arthropod is an invertebrate animal with an exoskeleton (outer skeleton), a segmented body and coupled articulated appendages. Arthropods form the phylum Euarthropoda, which includes insects, arachnids, myriapods and crustaceans. The term Arthropoda as originally proposed refers to a proposed group of Euarthropods and the phylum Onychophora. Arthropods are characterized by their articulated limbs and the cuticle made of chitin, often mineralized with calcium carbonate. The plane of an arthropod's body consists of segments, each with a pair of appendages. The rigid cuticle inhibits growth, and consequently arthropods replace it periodically by shedding.

The exoskeleton of arthropods is a coating apparatus and therefore presides over protection, secretion and excretion functions.

The basic structure of the exoskeleton is that of any complex tegumental system. From inside to outside, the exoskeleton consists of the following three layers: a connective tissue; an epithelial tissue, called epidermis; an inert tissue called cuticle.

The external cuticle is a tissue formed by the complex structural organization of macromolecular organic compounds that together must give the exoskeleton stiffness, elasticity and chemical inertia. The external cuticle is free of cellular elements, therefore it can be defined as an inert tissue.

The functional complexity of the cuticle is due to the differentiation of several layers, in which both the chemical composition and the structural organization change. The stratification sees the succession of two layers: epicuticola, more external, and procuticola, more internal.

The epicuticola, with a maximum thickness of a few microns, mainly performs functions of isolation and protection of the body from the external environment, its main properties are, therefore, chemical inertia and impermeability. From a chemical point of view, the epicuticola is of a waxy-lipoprotein-tannic nature. The waxes are localized in the outermost layer, whereas the lipoproteins are located inside.

Some arthropods are known infestants of the human body and animals. Infesting arthropods have been divided into:
temporary ectoparasites, i.e. those that remain on the body of the host for a limited period, then abandon it after a certain period of time (such as fleas, bedbugs, ticks);
obligatory ectoparasites, i.e. arthropods that live constantly on the body of the host.

Pediculosis, or phytiriasis, is a parasitic infestation caused by some species of louse (Pediculus), in particular infestation of the scalp due to Pediculus humanus capitis, characterized by the presence of the parasite itself and its eggs, called ‘nits’, that the parasite cements to the hair. It is generally very difficult to detect parasites and their eggs in suspected areas of infestation, especially due to their tiny size.

There are three types of lice that infest the human body, namely Pediculus humanus capitis, which live on the scalp causing pediculosis, Pediculus humanus corporis that inhabit the body and Phthirus pubis, or crab lice, that cause pubic pediculosis, all belonging to Anopluri.

Pediculosis is particularly common among school-age and pre-school children (4-13 years).

In the last twenty years there have been no significant innovations in the field of the treatment of pediculosis and the number of cases reported has increased to reach hundreds of millions of cases worldwide each year.

The methods already known treat pediculosis according to two different ways, i.e., chemically or mechanically.

According to the chemical method, the aim is to eradicate infesting lice and their eggs through the use of products containing antiparasitic agents as active ingredients in effective quantities such as pediculocides.

The products on the market based on antiparasitic agents are available in various forms including emulsions, gels, lotions and shampoos with rinsing and foams.

The main products on the market are natural pyrethrins (Milice®); synthetic pyrethrins, such as, for example, permethrin (Paranix, Mom® emulsion) and tetramethrin (Mom® powder and shampoo); phosphoric organic antiparasitic agents, such as malathion (Aftir®); lindane (γ-hexachlorocyclohexane).

The action mechanism of pyrethrins is to block the repolarization of sodium channels in the nerve cells of lice, causing death by paralysis. Malathion causes an accumulation of acetylcholine at the receptor level with subsequent respiratory paralysis of the louse. Respiratory paralysis is also the action mechanism of lindane.

Antiparasitic agents, however, are often ineffective for eradicating treatment. One of the main causes of the failure of treatment with these agents is the onset of strains of infesting arthropods resistant to the molecules of the antiparasitic agents used. The pathogenesis of this pharmacological resistance is multifactorial and is due to factors including incorrect use of pediculocides with reference to indication, dosage and duration of treatment (to which children are evidently exposed); type of formulation; residual pediculocidal effect; prophylaxis treatment not adequately performed.

Furthermore, antiparasitic agents such as lindane and pyrethrin act by blocking the nervous system of arthropods. As the central nervous system of lice develops only 3 or 4 days after hatching, only 60% or 70% of the parasites are eliminated by application of these antiparasitic agents. The National Pediculosis Association (USA) has indicated that between 20% and 80% of nits survive treatment with conventional antiparasitic agents, which results in a new infestation of the subject. Cases of contact dermatitis and allergic reactions have been reported in literature.

Furthermore, all of these products are absolutely not recommended for prolonged use due to their toxicity.

A repetition of the early and regular treatment of antipediculosis is however essential for completely eradicating the parasite and interrupting its life cycles.

U.S. Pat. No. 5,547,665 describes a complex disinfestation method comprising the application of an antiparasitic agent followed by treatment with an aqueous composition comprising an enzyme (N-acetyl-D-galactosaminidase) capable of breaking down the organic structure which cements the nits to the hair, which, however, must be followed by an accurate mechanical removal of the nits and parasites.

U.S. Pat. No. 4,927,813 describes the use of an aggressive chemical product (10% formic acid solution in water) which, however, does not remove more than 94% of lice, consequently it does not eliminate the risk of re-infestation.

U.S. Pat. No. 5,658,750 describes a colorimetric method for identifying ongoing infestations by the analysis of hair strands obtained from a subject, which is related to the determination of the vitality of parasites on samples of cut hair and does not concern the treatment or prevention of infestation, nor the elimination of nits.

U.S. Pat. No. 6,875,421 discloses a method for visualizing arthropods in parts of the body or in potentially parasitic environments, which comprises the application of an aqueous solution comprising from 0.01% to 10% by weight of a fluorescent substance to samples of hairs or hair or objects such as furniture, bed linen, brushes and combs.

U.S. Pat. No. 5,972,987 describes a method for visualizing lice and nits on the hair of a subject comprising the application of an alcoholic solution of a fluorescent substance, the mechanical removal of the parasites and the subsequent removal of the pigment from the hair. U.S. Pat. No. 5,972,987 indicates that the alcohol solution used is potentially irritating, therefore, contact with the face and skin of the person concerned must be avoided and whoever applies the solution must wear gloves and protective clothing in order not to come into contact with the alcohol solution of the pigment.

There are also antipediculosis products, defined as ‘natural’, based on the use of essential oils, in various associations, and which have so far only produced uncertain results.

The mechanical approach involves the removal of lice and nits from the head using thick-toothed steel combs, also available on the market in an electronic version that acts with a low-voltage current. This method is particularly effective in removing nits, firmly attached to the hair, but has the disadvantage of requiring an accurate and patient scrutiny lock by lock.

The removal is also particularly difficult because each nit is attached to a hair through a glue based on glycoproteins, which acts as a “cement” and the same lice eggs are covered with a sheath of chitin that surrounds both the nit and the hair.

Furthermore, it is extremely difficult to correctly effect a clean-up of all the nits present. Lice and nits, in fact, have a colour generally indistinguishable from that of the hair, and have small dimensions, consequently making it substantially impossible to detect parasites, and above all nits, with the naked eye.

Recently, the Nitview® Ledcomb device has been made available, which uses a phenomenon of autofluorescence of the chitin contained in lice and nits.

As is known, chitin is an organic substance, extremely resistant to chemicals, which is the exoskeleton of arthropods. Chemically it is a nitrogen-containing polysaccharide (C₈H₁₃NO₅)n, consisting of N-acetyl-D-glucosamine units joined via β-1,4 glycosidic covalent bonds to form long chains. Chitin consists of chains aggregated in laminas joined by hydrogen bonds according to an α, β or γ crystalline form on the basis of a corresponding aggregation mode.

The Nitview® Ledcomb device couples a microgrooved tooth comb, which facilitates the mechanical extraction of lice and nits, with an ultraviolet light emitting system. The chitin contained in lice and nits emits fluorescence when exposed to ultraviolet light.

The autofluorescence of chitin, however, is extremely low and consequently an accurate evaluation by the operator of the hair infested, strand by strand, is still required, with the possibility of error and inaccuracy, and ultimately remains unsatisfactory. In addition, exposure of the ocular surface to ultraviolet light can lead to eye damage, so the operator must wear protective glasses when using the Nitview® Ledcomb device.

As a consequence of the unsatisfactory efficacy of the antipediculosis treatment currently available, an increasing spreading of the problem of infestations due to lice has been recorded, especially in schools and environments frequented by children, and there remains a strongly felt need for an effective means of detecting and eliminating lice and their eggs.

The same applies to environments frequented by animals with fur, on which the presence of arthropods must be detected, such as, without limitation, lice and fleas, in order to both carry out immediate removal, and also to intervene with specific antiparasitic products.

An objective of the present invention is to provide a method for visualizing and removing arthropods from a human or animal subject, substantially free of the disadvantages of the methods illustrated above.

An object of the present invention relates to a method for detecting arthropods and/or their eggs in the hair of the body and head of a human subject or the fur of an animal subject by means of a composition in powder form comprising:

a) a fluorescent pigment suitable for selectively binding to the cuticle of arthropods and emitting fluorescence when exposed to visible or ultraviolet light radiation;

b) at least one starch or clay, preferably a starch;

wherein said method comprises at least the following steps:
i. applying the composition (C) to the hair of the head and/or body of the subject,
ii. exposing said hair of the body and/or head to a light source and
iii. detecting the arthropods, and/or their eggs, possibly present, through the fluorescence emitted.

In one embodiment, the present invention provides a composition (C) in powder form comprising:

a) a fluorescent pigment suitable for selectively binding to the cuticle of arthropods and emitting fluorescence when exposed to visible or ultraviolet light radiation;

b) at least one starch or clay, preferably a starch;

for use in the treatment or prevention of an infestation of a subject by arthropods, wherein said treatment or prevention comprises at least the following steps:
i. applying the composition (C) to the hair of the head and/or body of the subject,
ii. exposing said hair of the body and/or head to a light source,
iii. detecting the arthropods, and/or their eggs, possibly present, through the fluorescence emitted, and
iv. removing said arthropods, and/or their eggs, from the hair of the head and/or body of the subject.

An object of the present invention also relates to a composition (C) in powder form, the form of a dry spray comprising:
a) a fluorescent pigment suitable for selectively binding to the cuticle of arthropods and emitting fluorescence when exposed to visible or ultraviolet light radiation;
b) at least one starch or clay, preferably a starch;
c) at least one propellant.

Unless otherwise indicated, within the scope of the present invention, the percentage amounts of a component in a mixture are related to the weight of that component in grams with respect to the total volume of the mixture (in milliliters).

Unless otherwise specified, within the scope of the present invention, the indication that a composition “comprises” one or more components or substances means that other components or substances may be present in addition to that, or those, specifically indicated.

Unless otherwise specified, within the scope of the present invention, a range of values indicated for a quantity, for example the weight content of a component, includes the lower and upper limits of the range. For example, if the weight or volume content of a component A is referred to as “from X to Y”, wherein X and Y are numerical values, A can be X or Y or any of the intermediate values.

The method and composition according to the present invention can be used in human subjects or for veterinary use, for example, but without limitation, in pets such as dogs or cats, or in other mammals. The composition according to the present invention is preferably for use in human beings.

The inventors have surprisingly found that the combination of a starch or a clay and a fluorescent pigment, capable of binding to the chitin present on the outer layer of an arthropod, allows the preparation of a composition which is able to make the arthropods present on the hairs or hair of a subject, or on other parts of the body or on objects, more visible, which is particularly effective, easily applicable and fundamentally free of toxicity and side-effects, such as undesired staining of the skin and/or hair and clothing, encountered with the methods of the prior art for visualizing and eliminating parasites.

The objective of the present invention is to satisfy the requirements described above.

For this purpose, the present invention provides a precise and effective means for the detection of arthropods and specifically of lice and nits in visible fluorescence. The fluorescent pigment, by binding selectively and tenaciously to the external waxy (epicutic) layer of the exoskeleton of arthropods infesting human beings (lice, and nits) or animals (bugs and fleas), and not to the keratin of the hair or fur, or to the scalp, constitutes a detecting agent visible to the naked eye. Consequently, if lice, arthropods and/or nits are present in the treated area, they can be directly visualized and removed.

The fluorescent pigment in the composition (C) and (C1) according to the present invention is in powder form and is associated in a mixture with non-toxic natural substances, comprising at least one starch or clay in powder form, i.e. in the form of a granular solid finely divided into a plurality of particles which can flow freely, indicatively, but without limitation, having a particle size ranging from 1 to 100 micrometers. The pigment can be mixed with the starch or clay in powder form, or absorbed or adsorbed on said starch or clay in powder form.

In a preferred embodiment of the present invention, the pigment in the composition (C) and (C1) is at least one of: black iron oxide, yellow iron oxide, red iron oxide, Acid yellow 23 (CAS Nr. 1934-21-0), Food Yellow 3 (CAS Nr. 2783-94-0), Acid Red 15 (CAS Nr. 16423-68-0), Acid Red 14 (CAS Nr. 3567-69-9), Acid Red 18 (CAS Nr. 2611-82-7), Acid Blue 9 (CAS Nr. 3844-45-9), Acid Blue 74 (CAS Nr. 860-22-00) and mixtures thereof.

In the context of the present invention, starch refers to an organic compound of the class of carbohydrates (or glucide polysaccharide), commonly contained in food products such as bread, pasta, rice, potatoes, characterized by a large number of polymerized (+)-glucose units joined together by an α-glycosidic bond and consisting of 4/5 of amylopectin and 1/5 of amylose. The general crude formula of starch is (C₆H₁₀O₅)ₙ, wherein n is a variable number ranging from about one hundred up to some thousands and which indicates the residues of monomeric glucose units that are joined together to form the polymers, from which the various types of starches present in nature (e.g. rice starch, corn starch, etc.) derive.

In the context of the present invention, clay refers to a complex of mineral systems wherein the main element is aluminum silicate, it preferably refers to white clay (kaolin) or green ventilated and unventilated clay.

In a preferred embodiment of the present invention, the starch or clay in the composition (C) and (C1) is in a quantity ranging from 0.5 to 15%, more preferably from 4 to 10%, by weight/total volume of the composition.

In a preferred embodiment of the present invention, the starch in the composition (C) and (C1) is selected from rice starch, corn starch, potato starch, tapioca starch and mixtures thereof. Said starch is more preferably rice starch or corn starch.

In a preferred embodiment of the present invention, the starch in the composition (C) and (C1) is in a quantity of 4.5 to 8% by weight/total volume of the composition.

Without limitation, the starch b) and pigment a) can be in a ratio of 0.5 to 3% by weight/weight, preferably from 1% to 2% by weight/weight.

The starch, as well as the clay, in the composition (C) and (C1) according to the present invention, does not exert a viscosizing action, i.e. thickener, but rather serves to allow the link between the starch association (or clay) and fluorescent pigment and chitin present on the outer layer of the arthropod or nit.

In a preferred embodiment of the present invention, in the composition (C) or (C1), the weight of the sum of the pigment (component a)) and of the starch or clay (component b)) ranges from 1 to 15% of the total volume of the composition.

The present invention is directed, in particular, towards use for antipediculosis treatment, but is suitable for use against any other small infesting arthropods, such as for example bedbugs, fleas and mites, as these arthropods have a waxy layer-coated chitin exoskeleton (epicuticola).

The powder composition (C) for use in the method of the present invention can be, without limitation, in the form of dry powder or aerosol or dry spray.

In a preferred embodiment, said composition (C) is in the form of a dry spray, i.e. a spray substantially free of water (water less than 0.5% by weight/total weight of the composition) and comprises a fluorescent pigment. suitable and in an effective quantity for selectively and tenaciously binding only to the outer layer (exoskeleton) of arthropods and nits and which emits fluorescence under exposure to visible light radiation, for the detection of said arthropods and nits on the hair of the body or head of a human subject or of an animal with suspected infestation.

In a preferred embodiment of the present invention, the composition (C) or (C1) further comprises at least one of an alcohol, a silicone, a plasticizer and mixtures thereof.

In a preferred embodiment of the present invention, the alcohol in the composition (C) and (C1) is in a quantity ranging from 1 to 15%, more preferably from 6 to 9% by weight/total weight of the composition.

Said alcohol is preferably ethyl alcohol.

Said silicone, i.e. a polymer whose monomeric unit is composed of a silicon atom bound to an oxygen atom and to two alkyl or aryl radicals, is preferably at least phenyl trimethicone (CAS: 2116-84-9).

In a preferred embodiment of the present invention, the silicone in the composition (C) and (C1) is in a quantity ranging from 0.05 to 5%, more preferably from 0.1 to 3% by weight/total weight of the composition.

In a preferred embodiment of the present invention, the plasticizer in the composition (C) and (C1) is in a quantity ranging from 3 to 5% by weight/total weight of the composition. Said plasticizer is preferably isopropyl myristate.

In a preferred embodiment of the present invention, the composition (C) or (C1) further contains hair nutrients such as, without limitation, essential oils and vegetable oils, e.g. jojoba oil and argan oil.

In a preferred embodiment of the present invention, the composition (C) or (C1) further contains at least one perfuming agent such as, without limitation, Lavanda angustifolia essential oil or natural or synthetic extracts with peach, strawberry, lemon, vanilla fragrance and the like, or mixtures thereof.

In a preferred embodiment of the present invention, the composition (C) or (C1) further contains at least one antiparasitic active ingredient. For example, without limitation, said active antiparasitic agent may be at least one of natural pyrethrins, synthetic pyrethrins, such as for example permethrin and tetramethrin, phosphoric organic antiparasitic agents, such as malathion and lindane (γ-hexachlorocyclohexane). Said antiparasitic active ingredient is preferably a substance of natural origin substantially free of toxicity for animals other than arthropods, such as, without limitation, at least one of “tea tree oil” (Melaleuca alternifolia oil and similar species) and manuka oil (Leptospermum scoparium).

An embodiment of the present invention relates to a composition comprising components a) and b) as indicated above, dispersed in a pressurized gas c) and contained in a spray can in the form of a dry spray, i.e. containing less than 0.5% by weight of water with respect to the total weight of the composition.

In a preferred embodiment, said gas (c) is liquefied petroleum gas (LPG), propane, n-butane, i-butane or mixtures thereof. In a preferred embodiment, said gas c) is at a pressure of 2.7 to 3.2 bars.

A further object of the present invention relates to a kit comprising the composition (C1) as indicated above and a thick-toothed comb of the type commonly used for the removal of arthropods and their eggs, such as lice and nits.

There are numerous advantages of the present invention.

The operator is easily guided by the fluorescence to accurately recognize the position and number of arthropods, for example lice and nits, even in less accessible areas. It is therefore no longer necessary to search blindly strand by strand.

The mixture of starch or clay (component b)) and pigment a) used in the present invention is practically non-toxic, and it can therefore be used cyclically, even for control only.

This mixture does not affect either the hair or the scalp.

Furthermore, it is pleasant and fun to use, especially for children, thanks to its fluorescent colour.

The composition (C) according to the present invention not only allows arthropods and nits to be clearly and immediately detected and highlighted, but also eliminates the arthropod which dies suffocated by the action of the powder or by dehydration.

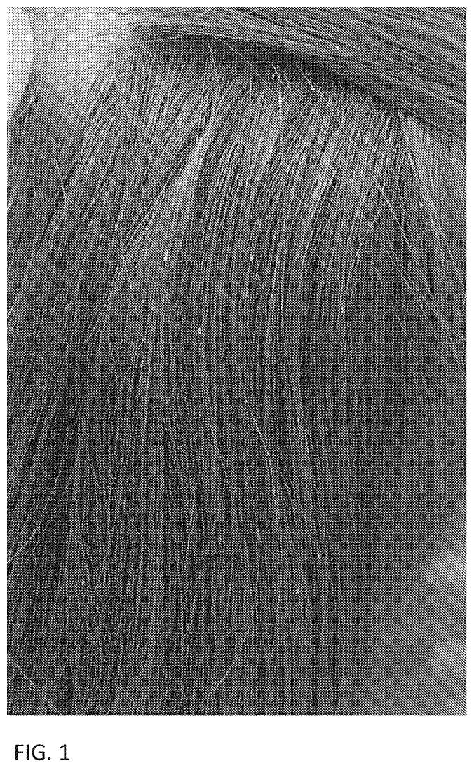

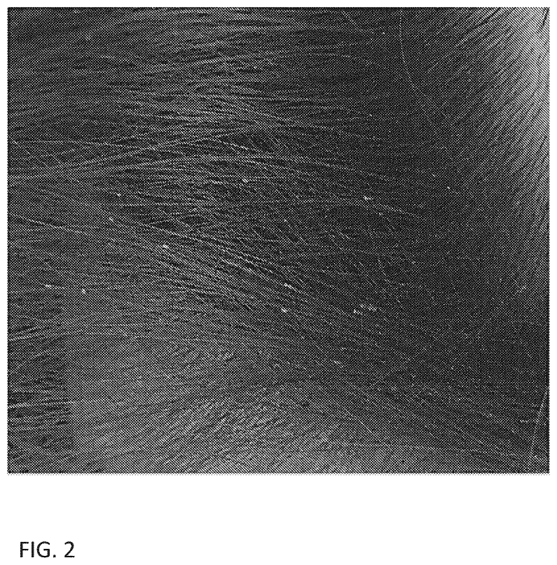

In FIGS. 1 and 2 of the present application, two subjects were photographed whose heads were subjected to treatment with the composition (C) according to the present invention: the presence of nits in the hair is clearly shown by the fluorescent powder which binds selectively and tenaciously to the nits (more evident in colour representations, but already very clear also from the black and white representations).

The following examples are provided in order to illustrate some embodiments of the invention, without limiting its scope.

In the present invention, the powder composition (C) can be in the form of a dry spray containing non-toxic natural substances and a suitable fluorescent pigment in an effective amount to bind to the outer layer which covers the chitin contained in arthropods coming into contact with these and emitting fluorescence under exposure to visible light radiation, for the detection of said arthropods in areas of the skin of a human subject or an animal with suspected infestation.

A particular method of application of the mixture object of the present invention is through the use of spray cans.

According to this preferred example, the powder composition is suitably dispersed in a gas and the whole mixture is compressed inside a cylinder, for example made of aluminum, for example from 150 ml, obtaining a dry spray shampoo.

According to a preferred example, the dry spray contains the ingredients present in Table 1 according to the volumetric percentages indicated.

TABLE 1

| | % WT./TOTAL |
|---|---|
| INGREDIENTS | WT. (% w/w) |
| Coloured starch | 5-9 |
| Antiparasitic agent (if present) | 3-5 |
| Alcohol | 6.3-9.3 |
| Fragrance | 0.1-0.3 |
| Silicone | 0.1-3 |
| Plasticizer | 3-5 |
| Gas | As necessary |

As indicated in Table 1, the volume of gas is variable and is what is necessary for filling the can, for example for a total volume of 150 ml of product.

According to a preferred example, the fluorescent pigment is pink (obtained by at least one of Acid Red 15 (CAS Nr. 16423-68-0), Acid Red 14 (CAS Nr. 3567-69-9), Acid Red 18 (CAS Nr. 2611-82-7 and mixtures thereof).

The colour of the mixture is white/transparent and the fluorescence is yellow, blue or pink (fuchsia). The colouring time ranges from 30 seconds to 5 minutes, the excess colour can then be removed with a brush or by simply rubbing the area on which the dry spray shampoo has been sprayed, with a cloth or with the hands.

No protection of the airways, eyes and ears is necessary as the dry spray or shampoo is completely non-toxic and, if it comes into contact with some of these parts, it is sufficient to remove it with the hands or with a cloth.

In the present invention, the composition according to the present invention can further contain hair nutrients such as essential oils and vegetable oils, in particular, but without limitation, jojoba oil or argan oil.

In the present invention the composition according to the present invention can further contain a perfuming agent. This can be for example Lavanda angustifolia essential oil or natural or synthetic extracts with peach, strawberry, lemon, vanilla fragrance etc.

In one embodiment, the present invention also provides a kit comprising a composition (C) or (C1) as described above, accompanied by a thick-toothed comb, for example a thick-toothed steel comb suitable for the removal of arthropods. and/or their eggs, for example of the type commonly used for the removal of lice and nits.

A further significant advantage of the composition according to the present invention is that it does not dye or stain and can therefore be removed very easily from the skin as it is in powder form.

### EXAMPLE 1

A composition according to the present invention having the composition indicated in Table 2

TABLE 2

| | % by wt./total |
|---|---|
| Component | wt. (% w/w) |
| Isopropyl myristate (IPM) | 4.0 |
| Phenyltrimethicone (DOW 556) | 0.5 |
| Ethyl alcohol | 7.3 |
| Fragrance (VELVETY PEACH TSV00060) | 0.2 |
| Coloured starch | 8.0 |
| LPG | 80.00 |
| | 100 |

in the form of dry spray contained in a 150 ml spray can was applied by spraying it on dry hair attacked by Pediculus humanus capitis and left to act for a couple of minutes during which the fluorescent detector adhered tenaciously to the waxy layer that covers the chitin of lice and nits. The hair was then brushed to remove the excess product. The mixture, especially if an antiparasitic agent is present, can help to kill lice, for example by dehydration, from the moment of application. The lice and nits were then identified with the naked eye, as the parasites and their eggs were clearly visible as fluorescent corpuscles on the non-fluorescent background of the hair, in an easy, unambiguous and immediate way. The lice and nits were removed mechanically with a thick-toothed comb until total elimination.

### EXAMPLE 2

The same composition used in Example 1, in the form of a dry spray contained in a 150 ml can, was applied by spraying it onto dry canine fur presumably attacked by Ctenocephalides canis and left to act for a few seconds, during which the fluorescent detector became bound to the outer waxy layer that covers the chitin of the fleas. The fur was then brushed to remove the excess product. The mixture, especially in the presence of an antiparasitic agent, can kill arthropods, for example by dehydration, from the moment of application. The fleas were then identified with the naked eye, and were clearly visible as fluorescent corpuscles on the non-fluorescent background of the fur, in an easy, unambiguous and immediate way. Once the presence of arthropods had been verified, it was possible to intervene both mechanically and also by acting with appropriate antiparasitic products until total elimination, verified visually.

### EXAMPLE 3 (COMPARATIVE)

A powder composition having the composition indicated in Table 3

TABLE 3

| | % by wt./TOTAL |
|---|---|
| Component | wt. (% w/w) |
| Disteardimonium hectorite | 3.0 |
| Isopropyl myristate (IPM) | 4.0 |
| Phenyltrimethicone (DOW 556) | 0.5 |
| Ethyl alcohol | 12.3 |
| Fragrance (VELVETY PEACH TSV00060) | 0.2 |
| Red dye | 0.01 |
| LPG | 79.99 |
| | 100 |

in the form of a dry spray contained in a 150 ml spray can, was applied by spraying it onto dry hair attacked by Pediculus humanus capitis and left to act for a couple of minutes.

The composition of Table 3 is characterized by the use of a clay, namely a derivative of hectorite, instead of starch. It is therefore a viscosifying agent not based on starch and the composition thus obtained, once applied to the hair, adheres tenaciously creating a creamy mixture. It is therefore not possible to remove the mixture from the hair by brushing and the composition also dyes the skin and mucous membranes. It is consequently not possible to selectively detect arthropods and nits.

The composition of Table 3 shows that the starch component is essential for the detection of arthropods and/or their eggs in the hair of the head and body of a human subject or in the fur of an animal subject.

### EXAMPLE 4 (COMPARATIVE)

A powder composition having the composition indicated in Table 4

TABLE 4

| | % by wt./total |
|---|---|
| Component | wt. (% w/w) |
| Isopropyl myristate (IPM) | 4.0 |
| Phenyltrimethicone (DOW 556) | 0.5 |
| Ethyl alcohol | 15.3 |
| Fragrance (VELVETY PEACH TSV00060) | 0.2 |
| Red dye | 0.01 |
| LPG | 79.99 |
| | 100 |

in the form of a dry spray contained in a 150 ml spray can, was applied by spraying it onto dry hair attacked by Pediculus humanus capitis and left to act for a couple of minutes.

The composition of Table 4 is characterized by the absence of the starch component and the alcohol-based composition is present at the moment of application in the form of aqueous droplets, it does not adhere to the hair and is not able to detect arthropods or parasites.

The composition of Table 4 shows that the starch component is essential for the detection of arthropods and/or their eggs in the hair of the head and body of a human subject or in the fur of an animal subject.

1. A method for the detection of arthropods and/or their eggs in the hair of the head and body of a human subject or fur of an animal subject by means of a composition, wherein the composition comprises:
a) a fluorescent pigment suitable for binding selectively to the cuticle of arthropods and emitting fluorescence when exposed to visible or ultraviolet light radiation;
b) at least one starch or clay;
wherein said method comprises at least the following steps:
i. applying the composition the hair of the head and/or body of the subject,
ii. exposing said hair of the head and/or body to a light source and
iii. detecting the arthropods, and/or their eggs, possibly present, through the fluorescence emitted.

2. The method according to claim 1, wherein the fluorescent pigment a) is selected from the group consisting of: black iron oxide, yellow iron oxide, red iron oxide, Acid yellow 23 (CAS Nr. 1934-21-0), Food Yellow 3 (CAS Nr. 2783-94-0), Acid Red 15 (CAS Nr. 16423-68-0), Acid Red 14 (CAS Nr. 3567-69-9), Acid Red 18 (CAS Nr. 2611-82-7), Acid Blue 9 (CAS Nr. 3844-45-9), Acid Blue 74 (CAS Nr. 860-22-00) and mixtures thereof.

3. The method according to claim 1, wherein the at least one starch is selected from the group consisting of corn starch, rice starch, potato starch, tapioca starch and mixtures thereof.

4. The method according to claim 3, wherein the at least one starch is selected from the group consisting of rice starch and corn starch.

5. The method according to claim 1, wherein the composition further comprises at least one of an alcohol, a silicone, a plasticizer, and mixtures thereof.

6. A composition in powder form comprising:
a) a fluorescent pigment suitable for selectively binding to the cuticle of arthropods and emitting fluorescence when exposed to visible or ultraviolet light radiation;
b) at least one starch or clay;
for use in the treatment or prevention of an infestation of a subject by arthropods, wherein said treatment or prevention comprises at least the following steps:
i. applying the composition to the hair of the head and/or body of the subject,
ii. exposing said hair of the body and/or head to a light source and
iii. detecting the arthropods, and/or their eggs, possibly present, through the fluorescence emitted, and
iv. removing said arthropods, and/or their eggs, from the hair of the head and/or body of the subject.

7. A powder composition in the form of a dry spray comprising:
a) a fluorescent pigment suitable for selectively binding to the cuticle of arthropods and emitting fluorescence when exposed to visible or ultraviolet light radiation;
b) at least one starch or clay, preferably a starch;
c) at least one propellant.

8. The composition according to claim 7, wherein the fluorescent pigment a) is selected from the group consisting of: black iron oxide, yellow iron oxide, red iron oxide, Acid yellow 23 (CAS Nr. 1934-21-0), Food Yellow 3 (CAS Nr. 2783-94-0), Acid Red 15 (CAS Nr. 16423-68-0), Acid Red 14 (CAS Nr. 3567-69-9), Acid Red 18 (CAS Nr. 2611-82-7), Acid Blue 9 (CAS Nr. 3844-45-9), Acid Blue 74 (CAS Nr. 860-22-00) and mixtures thereof.

9. The composition according to claim 7, wherein the at least one starch b) is selected from the group consisting of rice starch, corn starch, potato starch, tapioca starch and mixtures thereof.

10. The composition according to claim 7, wherein the weight of the sum of components a) and b) ranges from 1 to 15% with respect to the total weight of the composition.

11. The composition according to claim 7, wherein the composition further contains at least one antiparasitic active ingredient.

12. The method according to claim 1 wherein the at least one clay or starch is a starch.

13. The method according to claim 5 wherein preferably wherein the plasticizer is isopropyl myristate.

14. The composition according to claim 6 wherein the at least one clay or starch is a starch.

15. The composition according to claim 11, wherein the antiparasitic active ingredient is selected from the group consisting of natural pyrethrins, synthetic pyrethrins, phosphoric organic pesticides, and combinations thereof.

16. The composition according to claim 11, w herein the antiparasitic active ingredient is a substance of a natural origin substantially free of toxicity for animals other than arthropods selected from the group consisting of “tea tree oil” (oil of Melaleuca alternifolia and similar species) and manuka oil (Leptospermum scoparium).
